(19) United States
(12) Patent Application Publication
(10) Pub. No.: US 2020/0268470 A1
(43) Pub. Date: Aug. 27, 2020

(54) DEVICE FOR LIGHTING THE INTRAOCULAR CAVITY

(71) Applicant: PHARMPUR GMBH, Konigsbrunn (DE)

(72) Inventors: Christian Lingenfelder, Ulm (DE); Martin Hessling, Ulm (DE); Philipp Kölbl, Konigsbrunn (DE)

(21) Appl. No.: 16/609,077

(22) PCT Filed: Apr. 26, 2018
(86) PCT No.: PCT/EP2018/060817

§ 371 Date: May 4, 2020

(30) Foreign Application Priority Data
Apr. 26, 2017 (EP) 17168314.7
Apr. 27, 2017 (EP) 17168571.2

(51) Int. Cl. A61B 90/30; A61B 3/00; A61B 5/1455

(52) U.S. Cl. CPC A61B 90/30; A61B 2090/309; A61B 5/1455; A61B 3/0008

(57) ABSTRACT

An apparatus for illuminating an intraocular space in a human or animal eye in a targeted fashion is described, said apparatus comprising a) a base (2) with a proximal end and a distal end, b) a shaft (3) with a proximal end and a distal end, c) a probe (4) with a proximal end and a distal end, d) a light source (5) comprising at least one LED, and e) a power source (6) connected to the light source (5), wherein the shaft, at its distal end, is connectable to the proximal end of the base, the probe, at its proximal end, is connectable to the distal end of the base and wherein the light source (5) is positioned at the distal end of the probe (4).

### FIELD OF THE INVENTION

The invention relates to an apparatus for illuminating the eye as claimed in the preamble of claim 1.

### BACKGROUND TO THE INVENTION

Endoilluminators for illuminating the eye are known but have disadvantages. Thus, US 2004/0004846 A1, for example, has disclosed an apparatus that comprises an LED as a light source within a housing, this light source feeding light into an optical fiber that then illuminates the eye. A disadvantage of such an apparatus is that such apparatuses transport the light only very inefficiently from the light source to the location to be illuminated, and so only approximately 1% of the original light output arrives at the envisaged location.

Even though a good illumination of the eye or the fundus is necessary, care has to be taken that no phototoxic damage is caused. A good illumination is based on the color temperature, the emission characteristic and the brightness of the emitter in the eye. The power arriving at a certain area of the fundus emerges from these factors. Bright and high-energy light has a high power and may cause damage to the retina, the photoreceptors and the optic nerve. Although blue light is preferred for surgery since the surroundings in the eye are red and blue light facilitates an improved perception of the contrast, blue light is however higher energy light and therefore more likely to lead to damage. Expressed differently, a very good illumination of an operation region is therefore desired; however, the illumination should be as sparing as possible for the eye at the same time, both in view of the type of introduced radiation, which may represent a load on the tissue, and in view of the extent of the intervention on the eye. A high radiation exposure arises, in particular, if the light source comes too close to the retina and/or if the illumination duration is too long. The DIN standard ISO/DIS 15004-2 discloses the requirements for treatments on and in the eye. It is therefore important that not only the light source but also the distance thereof from the retina is defined in all cases since the radiant flux of the light source on a defined area of the retina becomes higher, the closer said light source is to the retina. This corresponds to a distance square law and, stated differently, expresses that the power incident on area A increases quadratically with reducing distance.

Distance Square Law:

$$E ∼ \frac{1}{{s}^{2}}$$

$$\begin{matrix}E & irradiance & W {cm}^{- 2} \\ s & working distance & mm \end{matrix}$$

In general, endoilluminators are used to illuminate the intraocular space in ophthalmic surgery and, in particular, in pars plana vitrectomy. The disadvantages of endoilluminators from the prior art should be considered to be that, in particular, the illumination is too low, or the radiation exposure of the eye is high, said radiation exposure being high as it is hardly controllable.

It was therefore an object of the invention to provide an apparatus for providing illumination for ophthalmic surgery, for example, for pars plana vitrectomy, said apparatus being able to illuminate the operating site in targeted and optimal fashion and protecting the sensitive regions of the eye, in particular the retina, to the best possible extent. Furthermore, it was an object of the invention to provide an apparatus which is flexibly adjustable, which allows the adaptation of, for example, color, intensity, direction and emission angle of the light and which facilitates an illumination at different wavelengths or an illumination with a combination of wavelengths. Furthermore, it was an object of the invention to provide an apparatus in which the emitted radiation of the light source lies within the scope of limits regulated by law or by guidelines. It was a further object of the invention to provide an apparatus that is compact and that can also be used for analysis in the eye under difficult external circumstances.

These objects are achieved by the apparatus as claimed in claim 1. In particular, an apparatus is made available for the targeted illumination of an intraocular space in a human or animal eye, said apparatus being very flexibly employable and able to be used in simple fashion. The apparatus can be adapted according to requirements. Moreover, the apparatus according to the invention not only offers the advantage that it is adjustable according to the respective use and for the respective requirements, but that the user is also able to undertake adaptations during use. In particular, the apparatus according to the invention can adapt parameters such as radiant intensity and color of the illuminated area to the respective use. Furthermore, various parameters of the radiation/light may also be altered as desired during use. The apparatus comprises a base, which has a shaft at its proximal end and a probe at its distal end. The probe is provided with a light source which, in turn, is connected to a power source. The light source comprises at least one LED but may also comprise a plurality of LEDs in order, for example, to set a desired color value. What is essential to the present invention is that the light source is positioned at the distal end of the probe.

Further advantageous embodiments are found in the dependent claims.

### FIGURES

FIG. 1 shows a sketch of an embodiment of the apparatus according to the invention for illuminating the intraocular space in a human or animal eye in a targeted manner.

FIG. 2 shows a sketch of a further embodiment of the apparatus according to the invention for illuminating the intraocular space in a human or animal eye in a targeted manner.

FIG. 3 shows a sketch of a further embodiment of the ophthalmic illuminator according to the invention.

FIG. 4 shows a sketch of the distal region of the ophthalmic illuminator according to the invention.

FIG. 5 shows a sketch of an LED suitable for use in the apparatus according to the invention.

FIG. 6A plots the irradiances of an LED according to the invention at 10 mA as a function of the wavelength of the emitted light at 0 mm distance from the detection area.

FIG. 6B shows the irradiance E_{A-R} with the evaluation function A(λ), a maximum exposure time tₘₐₓ(E_{A-R}), the irradiance E_{VIS-R} with the evaluation function R(λ) and the irradiance without the evaluation function (E₃₀₀₋₈₅₀).

FIG. 7 shows images of an illumination test using an ophthalmic illuminator according to the invention on a porcine eye.

### LIST OF REFERENCE SIGNS

1 Apparatus
2 Base
3 Shaft
4 Probe
5 Light source
6 Power source
21 Spacer
22 Distal end of the base
23 Proximal end of the base
24 Channel
30 Holder
61 Line
62 Current limiter
70 LED pen
72 LED pen base
73 LED pen shaft
74 LED pen probe
75 LED pen light source
76 LED pen switch
80 Trocar
81 Trocar holding part
82 Trocar cannula

### DEFINITIONS

The term “intraocular space”, as used in the present description, denotes the entire interior of the eye, in particular behind the sclera, and includes the fundus.

The term “base”, as used herein, relates to a holder that is suitable for receiving a probe on one side and for receiving a shaft on the other side.

The term “connectable”, as used herein, means the two parts are connectable or connected to one another. Any type of connection is suitable, with both permanent and temporary affixment being included. In particular, connectable means that one part may also be connected to another part at times and may be replaced by another part where necessary.

The term “integral” means belonging to a whole or forming a whole, i.e., parts or devices referred to as integral form a unit.

The expression “probe” denotes an apparatus that is rod-shaped and has at least one channel in the interior thereof.

According to the invention, use is made of a light source comprising at least one LED. The term “LED” comprises any type of LED known from the prior art, i.e., in particular, LEDs and OLEDs. The term “LED” includes LEDs with small dimensions, e.g., micro LEDs or nano LEDs. The term LED includes LEDs of any wavelength, i.e., LEDs that emit light with any wavelength from the UV to the far infrared and therebeyond.

The term “light in the visible range” is understood to mean radiation with a wavelength from approximately 0.38 to approximately 0.78 μm.

The term “light with a wavelength in the near infrared” is understood to mean infrared radiation with a wavelength from approximately 0.78 to approximately 2.5 μm and a wave number from approximately 12 500 to approximately 4000 cm⁻¹.

The term “light with a wavelength in the mid infrared” is understood to mean infrared radiation with a wavelength from approximately 2.5 to approximately 25 μm and a wave number from approximately 4000 to approximately 400 cm⁻¹.

The term “light with a wavelength in the far infrared” is understood to mean infrared radiation with a wavelength from approximately 25 to approximately 1000 μm and a wave number from approximately 400 to approximately 10 cm⁻¹.

The term “light with a wavelength in UV” is understood to mean UV radiation with a wavelength from approximately 0.2 to approximately 0.38 μm.

Here, the terms of “radiation” and “light” are used in the same way. If the term light is used, this should comprise radiation with a wavelength from UV to far infrared. Accordingly, the term light source relates to a source that emits radiation with a wavelength in the range from UV to far IR.

The term “light source comprising at least one LED” denotes light sources that comprise at least one LED but may also comprise a plurality of LEDs in combination. In particular, this term includes LEDs with any suitable embodiment and combinations of LEDs that are used to generate a desired color, in particular RGB LEDs. Moreover, this term also includes light sources that comprise at least one LED emitting infrared radiation, visible light or UV radiation, or a combination thereof, e.g., light sources that comprise a combination of LEDs that emit light in the visible range (approximately 0.38 to 0.78 μm) and those that emit light in the invisible range, i.e., infrared or UV range.

A “power source”, as understood in this case, is any apparatus that can supply power, more particularly a current, to the light source according to the invention in order to make the latter shine. In particular, a power source is an element that can supply an adequate amount of current for the operation of the apparatus in order to make the light source shine in controlled fashion. Examples of power sources are accumulators, batteries, capacitors, etc.

In one embodiment, the apparatus according to the invention has the form of a pen and is therefore referred to as “LED pen” in this description.

In the present description, the term “distal” relates to regions distant from the user, e.g., the surgeon, while “proximal” relates to regions of the apparatus closer to the user.

### DETAILED DESCRIPTION OF THE INVENTION

The present invention provides an apparatus (1) for illuminating an intraocular space in a human or animal eye in a targeted fashion, said apparatus comprising a base (2) with a proximal end (23) and a distal end (22), a shaft (3) with a proximal end and a distal end, a probe (4) with a proximal end and a distal end, a light source (5) comprising at least one LED, and a power source (6) connected to the light source (5), wherein the shaft, at its distal end, is connectable to the proximal end of the base, the probe, at its proximal end, is connectable to the distal end of the base and wherein the light source (5) is positioned at the distal end of the probe (4). Consequently, the apparatus according to the invention is an ophthalmic illuminator that is suitable, in particular, for illuminating the eye.

The apparatus according to the invention, i.e., the ophthalmic illuminator according to the invention, is distinguished by being usable in a very flexible manner, being able to be adapted according to requirements, being able to illuminate the operating site in a targeted and optimal manner and, at the same time, protecting the remaining eye, in particular the retina. The ophthalmic illuminator according to the invention is therefore very well suited to ophthalmic surgery and analytics, e.g., for dyeing and removing membranes in the eye or for interventions at the fundus, e.g., at the retina, and for any type of pars plana vitrectomy. Furthermore, the ophthalmic illuminator according to the invention is also suitable for a differential diagnosis, e.g., for distinguishing between oxygen-rich and oxygen-depleted tissue or autofluorescent tissue. The apparatus according to the invention is also distinguished by being compact and requiring no external connections to a light source that could impair the surgeon. The apparatus is sterile or sterilizable and can be used repeatedly or else as a disposable product. As a result, it is employable everywhere, even under difficult conditions. Furthermore, it was shown that the limits prescribed in the aforementioned DIN standard could be observed by the apparatus according to the invention and that a sufficient exposure time is facilitated.

The base of the apparatus according to the invention is a part that serves to hold, receive or support a probe at the distal end and a shaft at the proximal end. The base may have an opening or a channel, with the opening preferably being round or substantially round and the channel possibly being straight or curved and the cross section of the channel preferably being round or oval. The outer part of the base can have any form; preferably, it is round or oval. The outer wall can have, e.g., a straight, conical or biconical embodiment. The opening in the base or the channel serves to guide one or more power lines therethrough. The base can have a receptacle for the shaft, can be connected to the shaft or can be part of the shaft. The base can serve to guide a probe therethrough or else receive a probe at one end. A shaft is attachable at the other end, the proximal end.

The shaft of the apparatus according to the invention can have any desired form. It serves to hold and/or direct the apparatus. By way of example, the shaft can have relatively large dimensions and store all the electronics and the power supply therein. By way of example, the shaft can be an integral part of a pen that has the required operating elements for radiation flux, hue, frequency, etc. on the outer sleeve.

The probe of the apparatus according to the invention is rod-shaped, with the cross section of the rod being able to have any desired form. The probe serves to hold or carry the light source and, optionally, to receive a line from the light source to the power source, e.g., a power line. The probe can be rigid or flexible; it can have a controllable manifestation, e.g., as a result of using shape memory alloys.

An essential property of the ophthalmic illuminator according to the invention is that the intraocular space can be illuminated in a targeted and optimal manner. This is achieved, firstly, by virtue of the light source being situated at the distal end of the apparatus or, expressed differently, by virtue of not being disposed within the probe or a shaft but instead forming the distal end of the probe, and, secondly, by virtue of light being radiated directly into the eye without being guided along a light guide. As a result, there are few losses and a high efficiency. Moreover, light can be steered directly to where it is needed by way of the movement of the probe. This also avoids the necessity of bringing light from a light source via a line; relatively rigid lines are required to this end and these are bothersome during an intervention.

A further important feature is that the light beam emitted by the light source can be set or guided by the user. This allows an illumination of precisely the region that the user, e.g., the surgeon, must see, while other regions of the retina can be spared. The light beam or the light cone can be directed by moving the shaft. Size, form and direction of the light cone can be altered by selecting and/or adapting the LED and/or the probe, and by directing.

The described properties of the apparatus according to the invention allow the apparatus to be used for many different purposes since the light beam can be metered and focused more precisely than in the case of devices known from the prior art. Moreover, it was found that the vitreous humor is sufficiently transparent not only to visible light but also to IR and UV radiation, and so sufficient amounts of radiation arrive at the desired location, both in the case of invasive and non-invasive transscleral use.

One option for varying an LED lies in setting the emission angle. The emission angle of an LED can be calculated using the following formula:

$$α = 2 * ( arctan ( \frac{d}{l * 2} ) )$$

where
α=emission angle
d=diameter of the object/space to be illuminated
l=distance of the light source from the illuminated area
arctan=Inverse function of the tangent function

Accordingly a suitable LED can be selected for the respective purpose. The emission angle can be altered, e.g., restricted by a reflector, the light beam can be scattered by a lens or the light cone can be widened or collimated by a lens. The direction of the light beam/light cone can be altered by virtue of appropriately embodying the region of the light source emitting the light cone. These adaptations are known to a person skilled in the art. The emission angle (aperture angle) α of the light source can also be set by an appropriate encapsulation of the LED. Suitably, the LED is selected in such a way that the eye is not subject or hardly subject to a thermal load.

A further option for influencing the incidence of light consists of coating some of the surface of the LED in such a way that no light or little light emerges at unwanted sites, and so the majority of the light can emerge through a desired region. Using this, the desired site can be radiated in even more targeted fashion during the illumination, with more sensitive sites then being excluded from illumination.

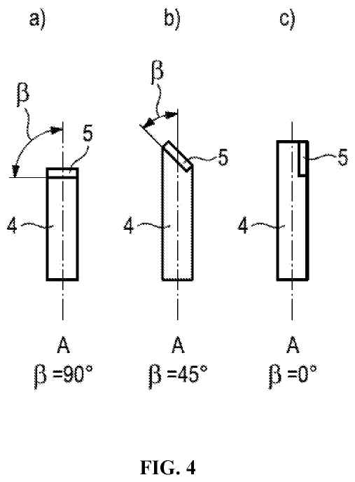

In a further embodiment, the light cone is influenced by virtue of the light source being attached to the probe at an angle β. The angle β in this case relates to the angle formed between the longitudinal axis (A) of the probe and the lower side of the light source. Thus, for example, the light source (5) can at an angle β of 0° or 5° to 90°, preferably 10° to 80°, preferably 20° to 70°, preferably 30° to 60°, preferably 40° to 50°, for example at an angle of 45° or 0°. In exemplary fashion, FIG. 4 shows an arrangement of the light source (5) at the probe (4) at an angle β of 90° (FIG. 4a), 45° (FIGS. 4b) and 0° (FIG. 4c).

By way of rotating or tilting, the light cone emitted by the light source can be directed in a targeted manner by hand by way of the shaft of the apparatus. A local alignment of the light cone by way of rotation is implemented, in particular, when the light source is not seated directly at the front of the probe but attached at an angle thereto. The local alignment can also be implemented by tilting the apparatus. To this end, the shaft in a preferred embodiment is embodied in such a way that it can easily be operated by the surgeon. By way of example, the shaft may have longitudinal ribs to increase its grip.

The simple operability, i.e., rotating or tilting, is obtained by virtue of the apparatus according to the invention being able to be “anchored” or held in tissue by way of the base. Expressed differently, the base can be pushed into tissue, in particular tissue in the pars plana. In this embodiment, the base can have a conical or biconical form in order to simplify “anchoring”. Here, “anchoring” is understood to mean that the base is pushed into a tissue region and remains there at said site. The probe of the apparatus according to the invention can also be guided in a trocar that has already been inserted into the tissue, with the trocar then providing the anchoring.

On account of the light source being disposed at the angle β, an operating site can be illuminated in optimal and tissue-sparing fashion at sites that cannot be reached by known devices, e.g., regions in the vicinity of the insertion site or at the periphery of the retina. On the other hand, a spacer provided at the base can prevent the probe with the LED from coming too close to the sensitive retina.

Thus, it is possible to direct the light cone in the eye on the site to be illuminated in extremely targeted fashion. Additionally, the illumination of sensitive sites such as, e.g., the macula can be excluded from the illumination in a targeted manner. At the same time, the targeted direction and restricted size of the light cone avoids further tissue being unnecessarily exposed to light and heat radiation.

The LED can be encapsulated by any material suitable for this type of light source, in particular transparent material. By way of example, a transparent glass, ceramic or polymer such an epoxy resin, acrylic resin, Perspex, polymethylmethacrylate, polyurethane or silicone is suitable. The choice of material determines the optical quality, which, as a rule, is higher for harder materials such as glass, and the tissue compatibility, which tends to be higher for softer materials such as silicone. Use can also be made of a material that acts as a diffuser or that modifies light as desired by diffraction.

The light source can have a plane, concave or convex surface with a round, rounded off or polygonal cross section. Advantageously, the edges of the light source or of the encapsulation of the light source are rounded off or beveled in order to avoid mechanical stimuli and splintering during the use of the ophthalmic illuminator on the eye.

Both encapsulating material and encapsulating form can be chosen in a targeted manner in order to influence light guidance, emission direction and light quality of the light source.

The apparatus according to the invention can furthermore comprise a controller, by means of which the color temperature and/or wavelength and/or brightness and/or intensity of the light is adjustable. The controller can have a conventional embodiment. By way of example, this may relate to a wireless interface that can be controlled by way of a remote control. Any possible type of control is possible here, e.g., control by speech or movement as well.

In order to set the color temperature of the light, one LED with the desired color temperature or a combination of two or more LEDs, which can then form the desired color, can be chosen in a targeted manner. A light source denoted an RGB LED is one example, said light source consisting of three LEDs connected in succession, which allow very many desired colors to be mixed. A change in the color temperature can be advantageous, for example in order to be able to better identify specific tissue structures under specific light, in order to be able to better identify and use operating devices under special light, in order to better identify specific dyes used for staining structures, or in order to be able to set the color temperature specifically for a user. Thus, for example, the wavelength can be matched to employed dyes, color pigments or nanoscale fluorescing particles or liquids before and/or during an operation.

Furthermore, the brightness of the light source can be set by way of targeted switching with different currents.

The controller that renders the color temperature of the light and/or the brightness of the light adjustable can be embodied as a switch with different switching levels. In another embodiment, switching is implemented electronically, e.g., by way of a voice control unit or a remote control. The latter can be present externally or integrated into the shaft.

Setting the parameters of emergence angle of the light, emission angle of the light, color temperature and brightness allows the best possible illumination of the operating site in the eye, while simultaneously sparing the affected and the adjoining tissue to the best possible extent and protecting the macula to the best possible extent.

An apparatus that is particularly well-suited to the respective purpose can be designed from the various parts of the apparatus according to the invention. Essential parts to this end are the probe, the base, the shaft and the light source. All parts of the apparatus can be produced from materials that are conventional for this application. The respective material should be biocompatible and sterilizable.

The base serves to hold and/or anchor the apparatus in tissue during intraocular use and to hold the probe on the distal side and the shaft on the proximal side. To this end, the base is embodied as a part with an opening. The base can be produced from conventional biocompatible and preferably also electrically insulating materials. By way of example, the base can be made of tissue compatible elastomeric materials, e.g., elastomeric silicone, elastomeric polymers, etc. Then, the base can adapt to the surroundings by way of a certain amount of elasticity. However, the material must not be too flexible since the passage, i.e., the opening, should be accessible. In the case of suitable forming, a thermoset is also a suitable material for the base. Moreover, the base can serve to keep the apparatus at a desired site in the tissue, e.g., in the region of the pars plana, and at a suitable distance.

A probe can be positioned at the distal side of the base. The probe serves to carry the light source at its front side and to admit in its interior the connection between the light source and the power source. To this end, use is made, as a rule, of connections, e.g., wires or a conductive polymer, which connect the light source to the power source situated at the proximal side of the base. The probe should be as thin as possible in order to damage as little tissue as possible. The material of the probe should be electrically insulating.

The thickness of the probe preferably ranges from 20 to 30 gauge, particularly preferably 23 to 27 or 28 gauge.

A further essential part of the apparatus according to the invention is a shaft that is positioned at the proximal side of the base. The shaft can either be securely connected to the base or else the base can be embodied to be able to receive and hold the shaft. The shaft serves for the operation of the apparatus, i.e., the positioning, rotating and tilting of the latter. Therefore, in one embodiment, the shaft is embodied in such a way that it comprises gripping depressions, gripping ribs, gripping areas or the like to allow it to be easily rotated and held by the user. The shaft can be produced from any possible material provided said material is biocompatible. Preferably, sterilizable and/or insulating material is used for the shaft. By way of example, sterilization can be implemented by gas and/or gamma rays.

For a simpler insertion, the apparatus according to the invention may still comprise a handle in addition to the shaft in one embodiment, said handle being positionable at the shaft or at the base in removable fashion. The handle can be removed after the apparatus has been inserted. It is also possible for a handle to be initially positioned at the base for the purposes of inserting the apparatus and the handle then to be replaced by the shaft following the insertion. As a rule, the handle is larger than the shaft and could form an impediment during an intervention, and so it is advantageous to remove said handle after use.

The light source (5) is electrically connected to a power source, with the light source being positioned distally at a probe while the power source is preferably disposed on the apparatus outside of the intraocular space, e.g., in the proximal region of the shaft. Light source and power source can be connected in contactless fashion or via electric lines. In one embodiment, the light source is connected to a power source (6) via a switch. The power source (6) may comprise one or more batteries, one or more accumulators, one or more capacitors or combinations thereof. In a preferred embodiment, the power source comprises at least one battery. Batteries as used for medical devices are suitable. Use is preferably made of lithium ion batteries or batteries or power sources with comparable properties, as these supply a very continuous current and consequently the luminous intensity of the light source remains fairly constant. A constant light intensity contributes to the advantageous properties of the ophthalmic illuminator according to the invention.

In order to further improve the stability of the emitted light and in order to keep the light intensity constant, a current limiter or an LED driver can additionally be used in a further preferred embodiment.

Preferably, the power source is situated in a housing provided to this end. The housing is preferably affixed in the vicinity of the proximal region of the shaft or is integrated into the shaft in order to provide an apparatus that is as compact as possible. The use of at least one accumulator or capacitor, which can be charged in contactless fashion or by a connection to a charging device, is also possible. Thus, the housing containing the power source and connected to the light source can, for example, be adhesively bonded in the vicinity of the apparatus, e.g., on the forehead of the patient, during an intervention.

The use of batteries or accumulators as power source facilitates the multifaceted use of the ophthalmic illuminator according to the invention.

In one embodiment of the apparatus according to the invention, the probe is fastened to the base and guided through the tissue via a holder and/or the shaft, for example in the region of the pars plana. In this embodiment, an opening in the tissue is produced first, e.g., by way of an incision, it then being possible to push the probe with the base therethrough.

In a further embodiment, the tissue is initially pierced with the aid of a usual puncturing instrument, e.g., a trocar. Subsequently, the probe according to the invention is then pushed into the intraocular space through said trocar. For this embodiment, the probe with the light source must have a smaller diameter than the trocar in order to be able to be pushed through. In this embodiment, the probe (4) is preferably configured with a reduced outer diameter, e.g., 18 to 30 gauge, preferably 23 to 27 or 28 gauge.

The above-described embodiments have many advantages over known apparatuses: they are simple in application, very flexible and can be adapted to very different circumstances. They can be inserted into the intraocular space using a minimally invasive technique and supply optimal illumination which, moreover, can still be adapted in respect of various parameters during the intervention.

In one embodiment of the ophthalmic illuminator according to the invention, all parts, i.e., base, shaft, probe, light source, power source and housing, are situated in fully assembled fashion and compactly in the housing; i.e., all constituent parts form a unit, are integrated and can be referred to as an integral apparatus. This embodiment is suitable for transscleral illumination. Here, the apparatus according to the invention can be embodied like a pen, a light source being situated at the tip thereof. The housing can have substantially the same outer diameter over its entire length. In particular, this embodiment can be used to illuminate the intraocular space in transscleral fashion. To this end, the apparatus can be guided in the conjunctival sac and illuminate the intraocular space through the sclera. The pen is guided by hand, i.e., the user can steer the light to precisely where it is required. The light passes through the sclera. This pen serves for diagnostic purposes, for illumination purposes and, simultaneously, as a denting implement. In this embodiment, the retina is illuminated from the outside and tears and holes become easily visible to the user. This embodiment is therefore well-suited to a noninvasive examination. A further advantage of this apparatus is that there is no need for wires, connections or holders extending to the outside. The pen can be produced as disposable product. It is taken from the packaging, is sterile, is used, and then discarded. Therefore, it is well-suited for use in the field, in difficult areas, in military hospitals, etc.

In one embodiment of this pen-shaped apparatus, use can be made of a plurality of LEDs, e.g., RGB LEDs, as these have enough space in this case. Here, the color of the light can be set as desired in each case by the user.

In another embodiment, a proximal region (11) of the shaft is configured as a handpiece (42) for directing the light cone. The handpiece preferably has a greater outer diameter than the remainder of the shaft. In a further embodiment, the surface of the handpiece and/or of the shaft is modified in order to achieve a better grip for directing the shaft and hence the light cone. Alternatively, it is possible to attach a small handle in the form of a rod attached perpendicular to the shaft or any other protrusions on the handpiece in order to simplify fine directing.

Further, a holder (42) is attached in the proximal region of the shaft (11) in a further embodiment of the apparatus according to the invention. This holder serves for better handling, e.g. when inserting the ophthalmic illuminator according to the invention into an insertion introduced at the pars plana. The holder is attached to the shaft in such a way that it can be unlocked and removed as soon as it is in no longer required. Then, the shaft or the handpiece from the alternative embodiment remains and facilitates fine directing of the shaft or of the light cone.

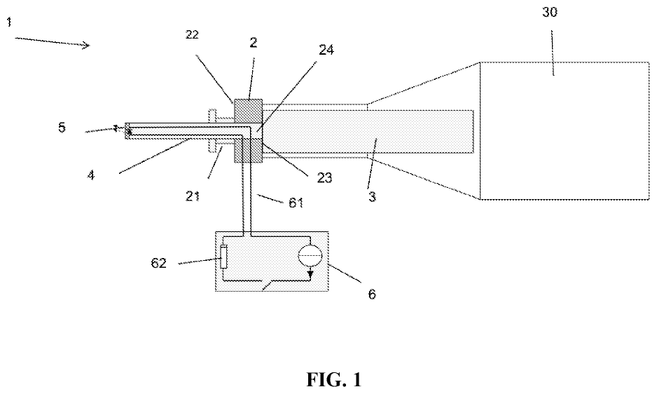

FIG. 1 shows an embodiment of the apparatus according to the invention for illuminating an intraocular space in a human or animal eye. The base 2 carries a probe 4 at the distal end 22. Moreover, situated on the base there is a spacer 21, which ensures that the probe cannot be pushed too far into the intraocular space in order thereby to prevent a radiation exposure that is too high or prevent injury by the probe. An LED 5 as a light source is situated at the distal end of the probe 4. The light source is connected by way of a line 61 to a power source 6 disposed outside of the apparatus. The line leads from the LED 6 through the probe 4 and a channel 24 in the socket 2 to the battery housing 6, in which optionally one or more batteries (not shown) are situated as a power source. Moreover, the battery housing 6 comprises a current limiter 62. A shaft 3 provided for directing the light cone when the apparatus is activated for illumination purposes is situated at the proximal side 23 of the base 2. A holder 30 is plugged onto the shaft 3, said holder serves for the application of the apparatus and can be removed as soon as the apparatus is anchored in the tissue.

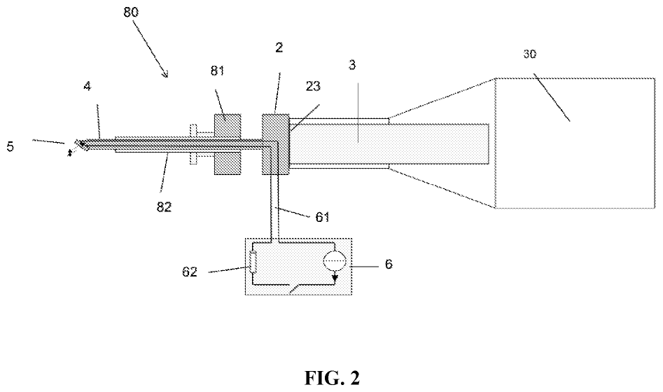

A further embodiment of the apparatus according to the invention is illustrated in FIG. 2. The apparatus illustrated in FIG. 2 for illuminating an intraocular space in a human or animal eye in a targeted manner comprises a base 2 that, at the proximal end 23, carries a shaft 3 suitable for directing the light cone. A holder 30 is plugged onto the shaft 3, said holder serves for the application of the apparatus and can be removed as soon as the apparatus is anchored in the tissue. At the distal end 21, the base 2 carries a probe 4, an LED 5 being positioned at the distal end thereof in such a way that said LED forms an angle β of 45° with the axis of the apparatus. From the LED 5, wires 61 lead through a channel (not shown) in the probe 4 into the base 2 and out of the apparatus, via a channel provided in a manner disposed at an angle in the base, to a battery housing 6 containing one or more batteries (not shown). As a result, the LED 5 is connected to a power source, the battery housing 6. Moreover, the battery housing 6 comprises a current limiter 62. In the embodiment of FIG. 2, the probe 4 leads through a trocar 80 that is anchored in the tissue. The trocar 80 consists of a holding part 81 and a cannula 82. The cannula 82 serves to penetrate the tissue and to guide and hold the probe of the ophthalmic illuminator according to the invention. During use, the probe 4 of the apparatus according to the invention is guided through the channel formed in the trocar 80 and the cannula 82 into the intraocular space in order to provide intraocular illumination. Once illumination is no longer required, the apparatus can be withdrawn again, and other instruments can be inserted through the channel 82 of the trocar 80. Once illumination is necessary again, the probe can be inserted in turn. This embodiment is very sparing and flexible since an inserted trocar can be used for different purposes, the trocar serves as a stop to prevent the probe from being able to be guided too far into the ocular space and only one incision is required in the tissue for illumination and instruments. This advantageous embodiment makes it possible, in the case of ophthalmic illumination at the pars plana, to initially only introduce the instrument channel into the incision in the eye and then, in the second step, introduce the ophthalmic illuminator through the instrument channel into the eye in sparing fashion and also freely rotate said ophthalmic illuminator, without further friction at the sclera.

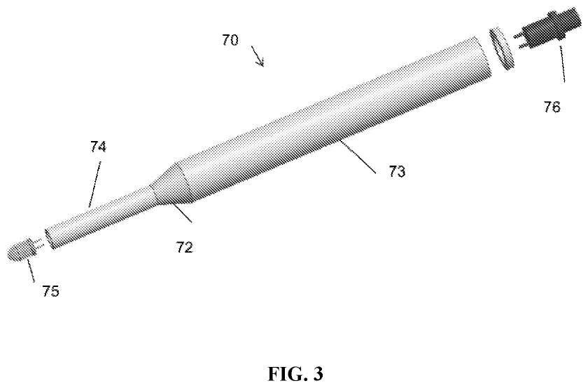

FIG. 3 shows a further embodiment of the apparatus according to the invention, which contains all components essential to the invention in a pen-shaped housing—an LED pen 70. This very compact embodiment, which requires no external sources for power or light, is ready for use anywhere. FIG. 3 shows a housing 70 with a probe 72, at the distal end of which a light source 75 (LED) is able to be plugged. A conical base 73 is situated between probe 74 and shaft 73. A switch that can be used to activate and deactivate the light source is able to be plugged to the proximal end of the shaft 73. A power source (not shown) that is connected via a line (likewise not shown) to the light source is situated in the housing. The LED of the pen can be guided in the conjunctival sac and illuminates the retina from behind such that the user can easily see tears and holes.

All embodiments were tested in the laboratory and met the required limits and allowed an illumination of the intraocular space to the desired extent.

The apparatus according to the invention is suitable for use in surgical and diagnostic methods on the eye, in particular for minimally invasive, intraocular illumination of the intraocular space in the human or animal eye.

The apparatus according to the invention is suitable for use in surgical and diagnostic methods on the eye, both for invasive and noninvasive methods; by way of example, it is suitable for noninvasive, transscleral illumination of the intraocular space in the human or animal eye.

For the noninvasive application, embodiments of the apparatus which have a light source with a rounded-off tip are suitable, in particular. The latter can also be used to exert targeted pressure on the retina in the case of the transscleral illumination in order, firstly, to improve the view and, secondly, to affix the retina, for example during the laser treatment.

On account of the many options for setting and directing the LEDs, for focusing and metering the light and for combining wavelengths, the apparatus according to the invention is usable in multifaceted ways. As a result of the structure of the apparatus and the position of the light source, light can be radiated on a selected site in targeted fashion and the luminous intensity and duration can be restricted. As a result of this, it is possible, for example, to use UV light for detecting fluorescing compounds in the ocular space without damaging the eye. By way of example, the detection of fluorescing compounds can be advantageous for detecting autofluorescent tissue or fluorescent markers.

The apparatus according to the invention can be advantageously used for obtaining diagnoses in the ocular space, e.g. for diagnosing tumors. Tumor tissue differs from “normal” tissue in terms of oxygen consumption and oxygen saturation. Oxygen-rich tissue can be differentiated from oxygen-depleted tissue by IR radiation. Since the apparatus according to the invention can be operated with infrared LEDs, it is possible by way of transscleral irradiation to examine the ocular space noninvasively and very sparingly for oxygen-rich tissue and hence contribute to the detection of tumor tissue and the regeneration of tumor tissue.

An advantage of all apparatuses according to the invention is that there is no need for a connection to a stationary device, in particular no need for a connection of optical fibers to a light source. The apparatus can be embodied as a pen that is preferably configured as a sterile disposable product. Therefore, the apparatus can be used everywhere, independently of the infrastructure, and it is easy to handle.

The invention will also be described and explained by the following examples, apparatuses and uses and methods, and constituent parts thereof, without being restricted thereto.

### EXAMPLES

### Example 1

### Current Supply by a Battery Reservoir

The current supply is facilitated by a battery reservoir. The latter can be equipped with a plurality of functions:
   simple switching for activating the illuminator
   switching with various fixed currents, e.g.:
      I=5 [mA]
      I=10 [mA]
      I=15 [mA]
      I=20 [mA]
      continuous control or control at discrete levels by a microcontroller
   control by way of a remote control

### Example 2

### Power Data of a Suitable LED

An LED suitable for use in the apparatus according to the invention has the following technical data:

Luminous color: White

Color coordinates: x=0.31 y=0.31

Luminous intensity: typ. 98 mcd

Color temperature: typ. 5500 K

Voltage: typ. 2.9 Volt

Current: max. 10 mA

Emission angle: 135°

Operating temperature: −40° C. to +85° C.

Soldering temperature: 5 seconds 260° C.

Dimensions: 0.65×0.35×0.2 mm

### Example 3

### Construction Data of a Suitable LED

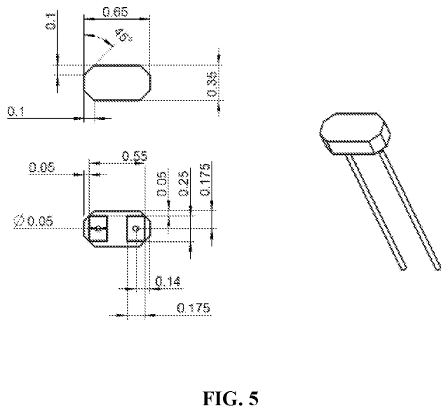

FIG. 5 shows an example of an LED suitable for use in the apparatus according to the invention. The LED illustrated in FIG. 4 has a plane surface with a polygonal cross section, in this case an octagonal cross section, and separated or rounded-off edges to avoid the edges splitting during the use on the eye.

### Example 4

### Irradiance of the LED

The irradiance of the entire LED was determined by means of an Ulbricht sphere and averaged values of a calibration lamp at the specified maximum current of I=10 [mA].

Table 1 plots the irradiances ascertained thus, which were measured at the distances of 0-18 mm.

TABLE 1

| | Irra- | | Photochemical | | |
|---|---|---|---|---|---|
| | diation | | hazard | | Thermal |
| | [E₃₀₀₋ | Light | Irra- | Max. | hazard |
| Distance | _{850 nm}] | flux | diation | exposure | Irradiation |
| [s] | μW | [ϕ_{V}] | [E_{A-R}] | time | [E_{VIS-R}] |
| mm | cm−2 | mlm | μW cm⁻² | [tₘₐₓ] h | μW cm⁻² |
| 0 | 1982.93 | 584.94 | 591.98 | 4.7 | 1972.24 |
| 1 | 385.07 | 107.76 | 127.51 | 21.8 | 383.10 |
| 2 | 163.14 | 47.80 | 49.50 | 57.9 | 162.25 |
| 3 | 76.30 | 22.31 | 23.26 | 120.8 | 75.89 |
| 4 | 44.79 | 13.02 | 13.78 | 204.6 | 44.55 |
| 5 | 26.51 | 7.68 | 8.22 | 338.5 | 26.36 |
| 6 | 16.89 | 4.88 | 5.23 | 533.0 | 16.80 |
| 7 | 12.21 | 3.51 | 3.83 | 727.5 | 12.14 |
| 8 | 9.37 | 2.70 | 2.93 | 949.4 | 9.33 |
| 9 | 7.46 | 2.11 | 2.43 | 1147.0 | 7.42 |
| 10 | 5.99 | 1.69 | 1.96 | 1425.0 | 5.95 |
| 11 | 4.80 | 1.35 | 1.57 | 1787.1 | 4.77 |
| 12 | 4.06 | 1.15 | 1.29 | 2156.1 | 4.04 |
| 13 | 3.44 | 0.97 | 1.13 | 2466.2 | 3.43 |
| 14 | 2.91 | 0.82 | 0.95 | 2959.0 | 2.90 |
| 15 | 2.56 | 0.72 | 0.86 | 3232.2 | 2.54 |
| 16 | 2.21 | 0.62 | 0.71 | 3903.4 | 2.20 |
| 17 | 1.92 | 0.54 | 0.61 | 4608.5 | 1.92 |
| 18 | 1.71 | 0.48 | 0.53 | 5232.7 | 1.71 |
| Limit for | | | 220.00 | | 350 000.00 |
| endo- | | | | | |
| illumin- | | | | | |
| ators | | | | | |

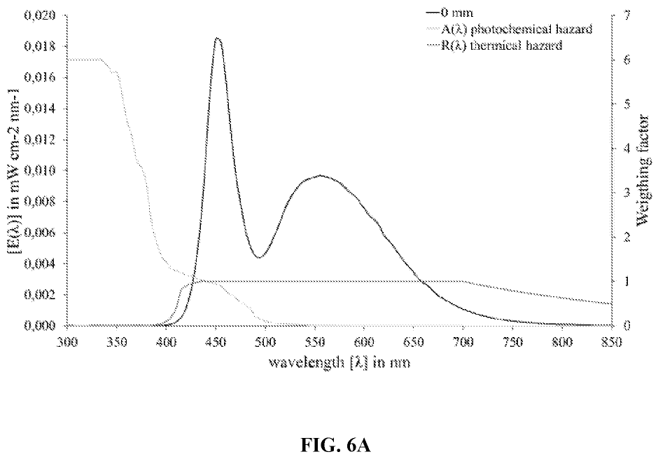

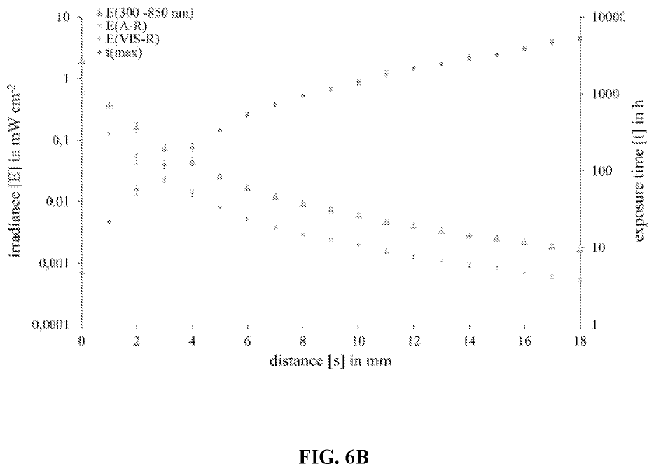

FIG. 6A plots the irradiances, measured in the case of 10 mA currents, as a function of the wavelength of the emitted light at a distance of 0 mm from the detection area. FIG. 6B shows the profile of the irradiance over increasing distance from the irradiation surface. In addition to the irradiance (300-850 nm) in the visible spectral range on an area of 1 mm, the profile of the weighted irradiance according to “Ophthalmic instruments—Fundamental requirements and test methods—Part 2: Light hazard protection (ISO/DIS 15004-2:2014); German version prEN ISO 15004-2:2014” can also be seen.

### Example 5

### Assessment According to ISO EN DIN 15000-2.2104

The LED was examined and assessed in respect of its photochemical hazard. The measured values were determined by means of an Ulbricht sphere and averaged values of a calibration lamp. The measured values were created by means of a 1 mm stop; the remaining evaluations were determined by calculation.

Table 1 represents the values, ascertained thus, as a function of the distance between the emitter and detector. The distance of 0 mm represents the value in the “worst case”. This would be the case if the light source were to touch the retina. Here, possible times of between 4.7 hours and 5232.7 hours arise, which, in turn, are realistic for a successful clinical application. The ascertained values show that no hazard to the eye tissue should be expected from the LED, chosen here, during the use in the apparatus according to the invention. Table 1 shows that limits in respect of the photochemical hazard were observed for all measured samples, even if there is no distance from the retina any more, i.e., when there is contact with the retina. However, contact with the retina should be avoided when the apparatus is used for examinations on the eye. Determining the maximum emission time tₘₐₓ from the ascertained irradiance E_{A-R} and the admissible overall amount of energy per unit area of 10 J/cm², pursuant to DIN EN ISO 15004-2:2014, shows that the light source is suitable for use in intraocular space.

The obtained values show that exposure times of far more than 30 minutes are possible for the apparatuses according to the invention. This allows widespread use in ophthalmic surgery and ophthalmic diagnostics.

### Example 6

### Test of the Micro LED Illumination on the Porcine Eye

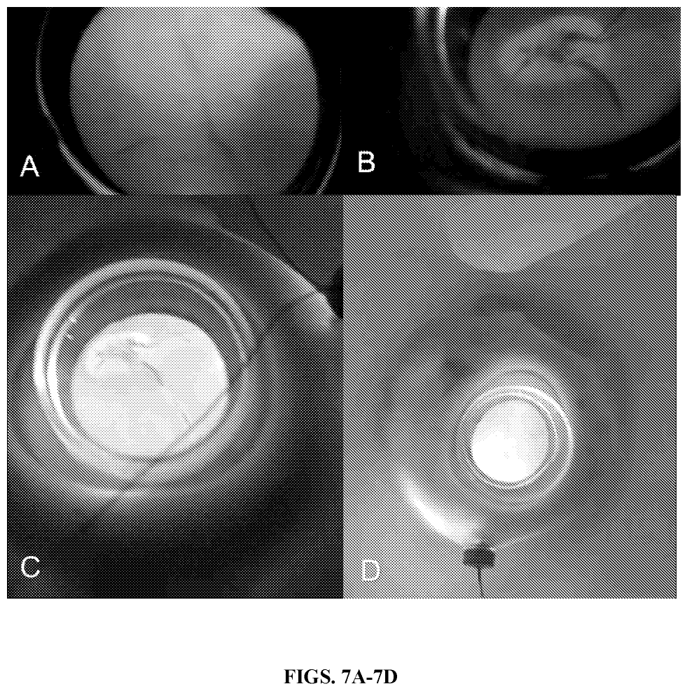

FIG. 7 shows images of an illumination test using an ophthalmic illuminator according to the invention on a porcine eye. A and B show images that were recorded using an ocular camera with a very low exposure time with a Zeiss surgical microscope. C and D were recorded using a Handycam and reflect the brightness sensitivity very well. In D, the cone of the LED did not point in the direction of the optic nerve, as can be identified from the bright site at the side of the eye.

1. An apparatus (1) for illuminating an intraocular space in a human or animal eye in a targeted manner, comprising:
a) a base (2) with a proximal end and a distal end,
b) a shaft (3) with a proximal end and a distal end,
c) a probe (4) with a proximal end and a distal end,
d) a light source (5) comprising at least one LED, and
e) a power source (6) connected to the light source (5),
wherein the shaft, at its distal end, is connectable to the proximal end of the base, the probe, at its proximal end, is connectable to the distal end of the base and wherein the light source (5) is positioned at the distal end of the probe (4).

2. The apparatus (1) as claimed in claim 1, characterized in that the light source (5) is disposed in such a way that an angle β in the range of 0° to 90° is formed between the lower side of the light source and the longitudinal axis (A) of the shaft.

3. The apparatus (1) as claimed in either of claims 1 and 2, characterized in that the base and the probe are integral.

4. The apparatus (1) as claimed in any one of the preceding claims, characterized in that the apparatus (1) comprises an interface that is connectable to a controller by means of which the wavelength, color temperature, intensity and/or brightness of the light are adjustable.

5. The apparatus (1) as claimed in any one of the preceding claims, characterized in that the light source comprises at least one LED that emits light with a wavelength in the near, mid or far infrared, emits light in the visible range or emits UV light.

6. The apparatus (1) as claimed in any one of the preceding claims, characterized in that the probe (4) has an outer diameter of no more than 20 to 30 gauge.

7. The apparatus (1) as claimed in any one of the preceding claims, characterized in that a proximal region (11) of the shaft is configured as a handpiece (30) for directing the light cone.

8. The apparatus (1) as claimed in any one of the preceding claims, characterized in that a holder (30) is attached in the proximal region of the shaft (11), wherein the holder (30) can be unlocked and removed.

9. The apparatus (1) as claimed in any one of the preceding claims, characterized in that the power source (6) comprises one or more batteries, one or more accumulators, one or more capacitors or combinations thereof.

10. The apparatus (1) as claimed in any one of the preceding claims, characterized in that the power source is integrated in the shaft in the proximal region of said shaft or in that the power source is integrated in a housing.

11. The apparatus (1) as claimed in any one of the preceding claims, characterized in that the apparatus (1) comprises a current limiter.

12. The apparatus (1) as claimed in any one of the preceding claims, characterized in that the apparatus (1) has an instrument channel that is not connected to the shaft, at least the distal region of said shaft being guided with rotational movement in said instrument channel, with the instrument channel extending at the shaft from distal to proximal and having an opening at the distal end for the emergence of the light.

13. The apparatus (1) as claimed in any one of the preceding claims, characterized in that the apparatus (1) comprises all constituent parts in integral fashion and/or said apparatus is embodied as a disposable article.

14. The apparatus (1) as claimed in any one of the preceding claims, characterized in that some or all components a), b), c), d) and e) of the apparatus (1) can be available in separate fashion and are combinable.

15. The apparatus (1) as claimed in any one of the preceding claims, characterized in that the distal end of the shaft is embodied in such a way that it does not damage the tissue when sweeping over the latter.

16. The use of the apparatus as claimed in any one of claims 1 to 15 for non-invasive, transscleral or minimally invasive, intraocular illumination of the intraocular space in the human or animal eye.

17. The use of the apparatus as claimed in any one of claims 1 to 15 for obtaining a diagnosis for the intraocular space in the human or animal eye.

18. The use of an apparatus as claimed in claim 5 for obtaining a diagnosis for oxygen-rich tissue, in particular for diagnosing cancer in the intraocular space in the human or animal eye.

19. A method for non-invasive, transscleral or minimally invasive, intraocular illumination of the intraocular space in the human or animal eye, wherein the method comprises the use as claimed in any one of claims 16 to 18.
